(19) United States
(12) Patent Application Publication
(10) Pub. No.: US 2016/0017273 A1
(43) Pub. Date: Jan. 21, 2016

(54) SPERM SELECTION UNIT STRUCTURE, SPERM SCREENING DEVICE PROVIDED WITH SPERM SELECTION UNIT STRUCTURE, AND METHOD OF PREPARING SEMEN FOR FERTILIZATION

(71) Applicant: NATIONAL INSTITUTE OF ADVANCED INDUSTRIAL SCIENCE AND TECHNOLOGY, Chiyoda-ku, Tokyo (JP)

(72) Inventors: Kenichi YAMASHITA, Saga (JP); Maria Portia NAGATA, Saga (JP); Masaya MIYAZAKI, Saga (JP); Hideaki MAEDA, Saga (JP)

(21) Appl. No.: 14/772,623

(22) PCT Filed: Jan. 6, 2014
(86) PCT No.: PCT/JP2014/050013

§ 371 Date: Sep. 3, 2015

(30) Foreign Application Priority Data
Mar. 5, 2013 (JP) 2013-043112
Oct. 22, 2013 (JP) 2013-219451

(51) Int. Cl. C12M 1/00; C12N 5/071

(52) U.S. Cl. CPC C12M 47/04; C12N 5/0612

(57) ABSTRACT

A sperm selection unit structure for efficiently separating sperm having favorable motility includes: a storage part which stores semen acquired from an animal therein; a buffer solution flow passage which communicates with the storage part and in which a buffer solution flows in the direction toward the storage part as a laminar flow; and a swim-up passage which communicates with the storage part such that the sperm swim up in the buffer solution flow passage from the storage part, wherein a downstream part of the swim-up passage is formed of a flow passage having a wide width where the buffer solution flows as a slow flow while maintaining a laminar flow state of the buffer solution, and a downstream end portion of the flow passage is opened in a state where the downstream end portion faces a wall surface of the storage part.

### TECHNICAL FIELD

The present invention relates to a sperm selection unit structure for selecting sperm having favorable motility from semen or the like in which sperm having no motility, dead sperm, deformed sperm and the like are mixed, a sperm screening device provided with the sperm selection unit structure, and a method of preparing semen for fertilization using the sperm screening device.

### BACKGROUND ART

Conventionally, in a livestock field, artificial insemination has been popularly performed for producing livestock. Also in infertility treatment of human or the like, artificial insemination has been performed in some cases.

In such artificial insemination, to make a female individual conceive a baby by injecting sperm obtained from a male individual into a female's uterus, semen having improved conception efficiency is desired.

For example, in the field of cattle, it is often the case that semen served for artificial insemination is semen frozen by liquid nitrogen after being taken out from an ox.

However, in semen which is defrosted after freezing, many sperm contained in semen are already dead or lost their motility, and a ratio of such sperm reaches approximately 50% to 70% in many cases. Further, such semen also partially contains deformed sperm, and this is considered as one of factors which obstruct the enhancement of conception efficiency.

Further, from dead sperm in semen, chemical species which should have been used when necessary in the course of fertilization are discharged at random.

As a result, sperm having favorable motility are also affected by these chemical species thus further lowering quality of semen and hence, the presence of dead sperm is not desirable.

Accordingly, with respect to semen used in artificial insemination, it is desirable that a ratio of sperm having favorable motility be high and dead sperm or the like be excluded from the semen.

In view of the above, conventionally, in the livestock field and the field of infertility treatment of human, there have been proposed various methods of recovering sperm having favorable motility.

Among these methods, the method of separating living sperm by making use of motility which the living sperm possesses imparts a relatively small burden on sperm and hence, it is safe to say that the method is useful as a means for separating and recovering sperm having favorable motility from sperm which have lost motility.

For example, there has been known a method where a fine flow passage (micro flow passage) is formed on a substrate having a size of a slide glass, a predetermined liquid such as a buffer solution is made to flow into the fine flow passage, and sperm having favorable motility are recovered by making use of a nature of the sperm that the sperm swim up against the flow of the buffer solution (see non-patent literature 1, for example).

According to such a method, it is possible to recover sperm having relatively favorable quality by reducing a damage imparted to sperm compared to a method where sperm are subjected to centrifugal separation or the like.

### CITATION LIST

### Non Patent Literature

NPL 1: Microfluidics and Nanofluidics, 2007, Vol. 3, 561-570

### SUMMARY OF INVENTION

### Technical Problem

However, in the above-mentioned conventional method of recovering favorable sperm by making use of a swim-up ability of the sperm, there is still a room for improvement from a viewpoint of efficiency of recovering sperm having favorable motility.

To be more specific, according to the description of non-patent literature 1, it is reported that only approximately ten pieces of sperm can be recovered per 1 minute.

For example, in performing in vitro fertilization (microscopic fertilization), there arises no problem when sperm can be obtained with such an efficiency. However, in the case of artificial insemination, that is, intra uterine insemination (IUI) or the like is performed, in an actual practice, it is necessary to remarkably enhance recovery efficiency (for example, ten thousand times to one hundred thousand times) by taking into account time necessary for separation of sperm or the like.

The present invention has been made in view of such circumstances, and it is an object of the present invention to provide a sperm selection unit structure capable or efficiently separating sperm having favorable motility.

It is another object of the present invention to provide a sperm screening device provided with the sperm selection unit structure, and a method of preparing semen for fertilization using the screening device.

### Solution to Problem

To overcome the above-mentioned problems of the related art, the present invention provides a sperm selection unit structure which includes: a storage part which stores semen before selection and containing sperm acquired from an animal therein; a buffer solution flow passage which communicates with the storage part and in which a buffer solution flows in the direction toward the storage part as a laminar flow; and a swim-up passage which communicates with the storage part such that the sperm swim up in the buffer solution flow passage from the storage part, wherein a downstream part of the swim-up passage is formed of a flow passage having a wide width where the buffer solution flows as a slow flow while maintaining a laminar flow state, and a downstream end portion of the flow passage is opened in a state where the downstream end portion faces a wall surface of the storage part.

The sperm selection unit structure according to the present invention also has the following technical futures.

(1) The flow passage having a wide width is configured to be narrowed toward an upstream side from the opening.

(2) A plurality of swim-up passages are connected to the storage part.

Further, in a sperm screening device of the present invention provided with the above-mentioned sperm selection unit structure where sperm acquired from an animal are made to swim up in a predetermined zone in a buffer solution in a laminar flow state, and the sperm which succeed in swimming up are recovered, a recovery passage which recovers the sperm which succeed in swimming up and a buffer solution supply passage which supplies a buffer solution which is made to flow down in the swim-up passage and the recovery passage are connected to an upstream end of the swim-up passage, a portion of the buffer solution supplied from the buffer solution supply passage is made to flow down into the swim-up passage, and a remaining portion of the buffer solution is made to flow down into the recovery passage thus forming a recovering flow, and the sperm which succeed in swimming up are recovered in the flow of the recovering flow.

In the sperm screening device according, to the present invention, the buffer solution supply passage has a flow passage cross-sectional area capable of supplying a flow rate of a buffer solution which enables the recovering flow to have a flow speed exceeding a swim-up ability of the sperm which succeed in swimming up through the re-covering flow.

A method of preparing semen for fertilization according to the present invention is directed to a method of preparing semen for fertilization using the above-mentioned sperm screening device, the method including: a sperm adding step of adding the semen before selection to the storage part; a sperm swim-up step of making sperm contained in the semen before selection added to the storage part swim up through the swim-up passage by making a buffer solution flow through the buffer solution flow passage and the recovery passage from the buffer solution supply passage at a flow speed which exceeds motility of the sperm which succeed in swimming up with respect to the recovery passage and at a flow speed which exceeds a limit flow speed by which sperm having motility but being not desired to be recovered are swimmable up and is lower than a limit flow speed by which sperm having favorable motility are swimmable up and at which a laminar flow is formed in the swim-up passage with respect to the buffer solution flow passage, and a recovery step of recovering the sperm which succeed in swimming up and are captured by the recovering flow thus preparing semen for fertilization.

In the method of preparing semen for fertilization according to the present invention, using timing at which sperm having a sex chromosome X and contained in the semen before selection acquire fertility as a threshold time, a ratio of sperm having a sex chromosome Y contained in the semen for fertilization is increased by performing the sperm adding step before the threshold time elapses, or a ratio of sperm having a sex chromosome X and contained in the semen for fertilization is increased by performing the sperm adding step after the threshold time elapses.

In the method of preparing semen for fertilization according to the present invention, in a sperm preservation step in which the semen before selection is preserved before the sperm adding step is performed, among sperm in the semen before selection, motility of sperm having either one of sex chromosomes out of the sperm having the sex chromosome X and the sperm having the sex chromosome Y is enhanced so that the swim-up of the sperm having either one of the sex chromosomes in the swim-up passage is accelerated in the sperm swim-up step whereby a ratio of sperm having either one of sex chromosomes and contained in the semen for fertilization acquired by the recovery step is increased.

In the method of preparing semen for fertilization according to the present invention, in a sperm preservation step in which the semen before selection is preserved before the sperm adding step is performed, among sperm in the semen before selection, motility of sperm having either one of sex chromosomes out of sperm having sex chromosome X and sperm having sex chromosome Y is lowered so that the swim-up of sperm having either one of sex chromosomes in the swim-up passage is suppressed in the sperm swim-up step whereby a ratio of sperm having the other sex chromosome and contained in the semen for fertilization acquired in the recovery step is increased.

In the method of preparing semen for fertilization according to the present invention, a preservation time in a sperm preservation step in which the semen before selection is preserved before the sperm adding step is performed is prolonged so that a ratio of sperm having sex chromosome X and contained in the semen for fertilization is increased.

In the method of preparing semen for fertilization according to the present invention, in a sperm preservation step in which the semen before selection is preserved before the sperm adding step is performed, a phenoxazine compound and an electron, accepting substance are present in the semen before selection so that a ratio of sperm having sex chromosome X and contained in the semen for fertilization is increased.

In the method of preparing semen for fertilization according to the present invention, a sperm preservation step in which the semen before selection is preserved before the sperm adding step is performed, a phenoxazine compound is present in the semen before selection so that a ratio of sperm having sex chromosome V contained in the semen for fertilization is increased.

In the method of preparing semen for fertilization according to the present invention, the electron accepting substance is at least one selected from a group consisting of flavins, phenazines, and NAD(P)H oxidoreductase.

In the method of preparing semen for fertilization according to the present invention, in the sperm preservation step, hexose-6-phosphate is further added to the semen before selection.

In the method of preparing semen for fertilization according to the present invention, the hexose-6-phosphate is one or a mixture of two or more selected from a group consisting of D-glucose 6-phosphate, fructose-6-phosphate, mannose-6-phosphate and galactose-6-phosphate.

In the method of preparing semen for fertilization according to the present invention, the phenoxazine compound is one or a mixture of two or more selected from a group consisting of brilliant cresol blue, nile blue, basic blue and meldola blue, and the hexose-6-phosphate is one or a mixture of two or more selected from a group consisting of D-glucose 6-phosphate, fructose-6-phosphate mannose-6-phosphate and galactose-6-phosphate.

### Advantageous Effects of Invention

The present invention can provide the sperm selection unit structure capable of efficiently separating sperm having favorable motility. The present invention can also provide a sperm screening device provided with a selection unit structure capable of efficiently separating sperm having favorable motility. The present invention further can provide a method of preparing semen for fertilization which contains a large amount of sperm having favorable motility.

### BRIEF DESCRIPTION OF THE DRAWINGS

FIG. 1A and FIG. 1B A conceptual view showing a sperm selection unit structure according to an embodiment.

FIG. 2A and FIG. 2B A conceptual view showing the sperm selection unit structure according to the embodiment.

FIG. 3A and FIG. 3B A conceptual view showing the sperm selection unit structure according to the embodiment.

FIG. 4 An explanatory view showing a sperm screening device according to the embodiment.

FIG. 5 An exploded perspective view showing the constitution of the sperm screening device according to the embodiment.

FIG. 6 A cross-sectional view showing the constitution of the sperm screening device according to the embodiment.

FIG. 7 An explanatory view showing the constitution of a micro flow passage.

FIG. 8 An explanatory view showing an area in the vicinity of a merging portion.

FIG. 9 An explanatory view showing a swim-up flow of sperm in a laminar flow passage.

FIG. 10 An explanatory view showing a state of swim-up sperm.

FIG. 11 An explanatory view showing a state of swim-up sperm.

FIG. 12 An explanatory view showing micro flow passages in chip bodies used in a test.

FIG. 13 An explanatory view showing a result of the test.

FIG. 14A and FIG. 14B An explanatory view showing a conventional example

### MODE FOR CARRYING OUT THE INVENTION

The present invention provides a sperm selection unit structure including: a storage part which stores semen before selection and containing sperm recovered from an animal therein; a buffer solution flow passage which is communicated with the storage part and allows a buffer solution to pass therethrough in the direction toward the storage part as a laminar flow and a swim-up passage which is communicated with the storage part such that sperm reversely flows in the buffer solution flow passage from the storage part.

Here, it is construed that the swim-up passage through which sperm swim up against the flow of a buffer solution is a portion substantially equal to the buffer solution flow passage through which a buffer solution supplied by a predetermined buffer solution supply unit flows in the direction toward the storage part as a laminar flow. That is, to observe the sperm selection unit structure according to this embodiment from a viewpoint of sperm which swim up, it is safe to say that the buffer solution flow passage is a portion which functions also as a swim-up passage.

Conventionally, the sperm selection unit structure which allows sperm to swim up by using the buffer solution flow passage as the swim-up passage as described above has been known as a method for performing the selection of sperm having favorable motility by making use of a property of sperm which is referred to as “rheotaxis”.

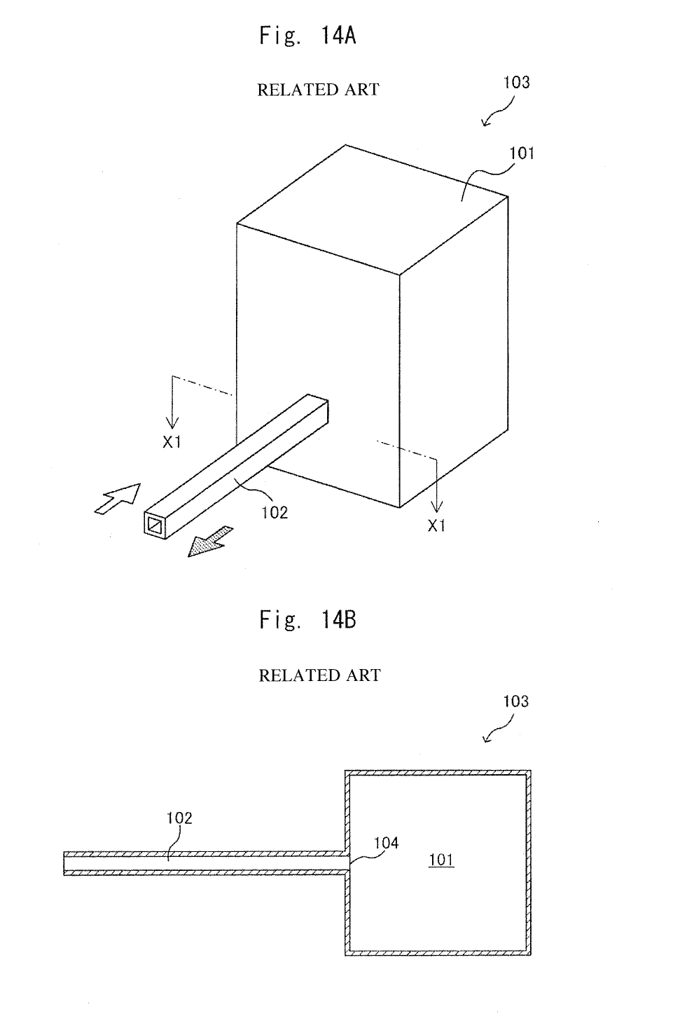

For example, as shown in FIG. 14A, a selection unit structure 103 where a narrow swim-up passage 102 is connected to a side wall of a storage part 101 which stores semen therein corresponds to the above-mentioned sperm selection unit structure.

According to such a selection unit structure 103, by making a buffer solution flow in the narrow swim-up passage 102 toward the storage part 101 as indicated by a blanked arrow, sperm can be guided into the swim-up passage 102 from an opening portion. 104 (see FIG. 14B) formed in an inner surface of the side wall of the storage part 101 thus allowing the sperm to swim up in the direction indicated by a meshed arrow.

However, in the conventional selection method which makes use of rheotaxis or swim-up of sperm and in the conventional selection unit structure which uses such a selection method, it is difficult to efficiently acquire a sufficient amount of sperm having favorable motility at the time of performing artificial insemination.

The reason is considered that the flow of a buffer solution which flows through the swim-up passage 102 at a relatively high flow speed flows into the inside of the storage part 101 from the opening portion 104 while maintaining its energy and hence, only sperm extremely excellent in motility can approach the opening portion 104.

Accordingly, such a selection unit structure 103 is useful for selecting an extremely small amount of sperm considerably excellent in motility. However, it is difficult, for a large amount of sperm having fertility to enter even the narrow swim-up passage 102 and hence, the population of sperm subjected to the selection is decreased whereby the above-mentioned selection unit structure 103 is not suitable to recover a large amount of sperm.

In view of the above-mentioned situation, the sperm selection unit structure according to this embodiment is characterized in that a downstream part of a swim-up passage is formed of a flow passage having a wide width (hereinafter, also referred to as a laminar flow passage) where a buffer solution flows as a slow flow while maintaining a laminar flow state, and a downstream end portion of the flow passage opens in a storage part in a state where the downstream end portion faces a wall surface of the storage part.

With such a configuration, the flow of a buffer liquid having a wide width and in a laminar flow state which is formed into a slow flow after passing the laminar flow passage can be made to flow out into the storage part from the opening. Accordingly, while keeping sperm having no motility or motes away from the slit-shaped opening. On the other hand, it is possible to induce a large amount of sperm having motility into the laminar flow passage by allowing the sperm to generate the property of rheotaxis while providing a frontage having a wide width to the sperm. Although the shape of the opening, is not particularly limited, for example, the opening may have the same shape as a cross-sectional shape of the laminar flow passage or may be opened in a slit shape on an inner wall surface of the storage part.

Further, the laminar flow passage is formed with a wide width while being formed into a shape where the laminar flow of a buffer solution which flows downwardly can be maintained. Accordingly, compared to a flow speed of a buffer solution which flows in a narrow flow passage portion of the swim-up passage (hereinafter also referred to as a narrow swim-up passage), it is possible to decrease a flow speed of the buffer solution which flows through the laminar flow passage without generating a turbulence in the buffer solution. Accordingly, the number of the population of sperm selected in the narrow swim-up passage can be enhanced.

To further describe the laminar flow passage, although the laminar flow passage is formed with a wide width compared to the narrow width swim-up passage, the laminar flow passage is formed into a shape by which the flow of a buffer solution having the small Reynolds number (for example, 1500 or below) can be formed. Accordingly, there is no possibility that sperm are entrained in the turbulence and hence, it is possible to efficiently induce sperm into the narrow swim-up passage.

In this manner, the sperm selection unit structure, according to this embodiment may be considered as the structure where a laminar flow passage which functions as an inducing means for efficiently inducing sperm into the narrow swim-up passage is interposed between the storage part for storing sperm therein and the narrow swim-up passage where sperm are actually selected.

That is, it is also safe to say that the sperm selection unit structure according to this embodiment is the sperm selection unit structure which includes: the storage part which stores semen before selection and containing sperm recovered from an animal therein; and the narrow swim-up passage where sperm are introduced from the storage part and are made to swim up in the laminar flow of a solution, wherein the inducing means for inducing sperm to the narrow swim-up passage from the storage part is interposed on a downstream part of the narrow swim-up passage, and the inducing means is a flow passage having a wide width which makes the flow of a solution slow while maintaining a laminar flow state of the solution which flows downward in the narrow swim-up passage, that is, a laminar flow passage.

Further, the laminar flow passage may be also considered as a stand-by region for sperm which form the population where sperm which exhibit the rheotaxis property are recovered as much as possible while allowing the presence of superiority or inferiority of motility among sperm to some extent and sperm is made to advance toward the narrow swim-up passage which forms a rapid flow compared to the laminar flow passage.

The laminar region may be also considered as a region for concentrating (aggregating) living sperm from semen before selection in the storage part while allowing the presence of superiority or inferiority of motility to some extent.

In performing the selection of sperm, semen before selection and containing sperm is stored in the storage part. As such a semen before selection. Not to mention that semen acquired from a male animal individual and preserved as it is, and semen which is thawed after being frozen can be used, semen where the concentration of sperm and components constituting seminal plasma are adjusted by adding seminal plasma and a buffer solution; and a sperm containing liquid prepared by dispersing sperm in a predetermined buffer solution can be also used.

That is, in this specification, semen before selection to be added to the storage part means, as a matter of course, semen acquired from a male animal individual and preserved as it is, semen before selection to added to the storage part is also a general term for thawed semen, component adjusted semen, and a sperm containing liquid.

Further, it is preferable that, in the sperm selection unit structure according to this embodiment, the laminar flow passage he configured to be gradually narrowed toward an upstream side thereof from the opening formed in the wall surface of the storage part, and be communicated with the narrow swim-up passage.

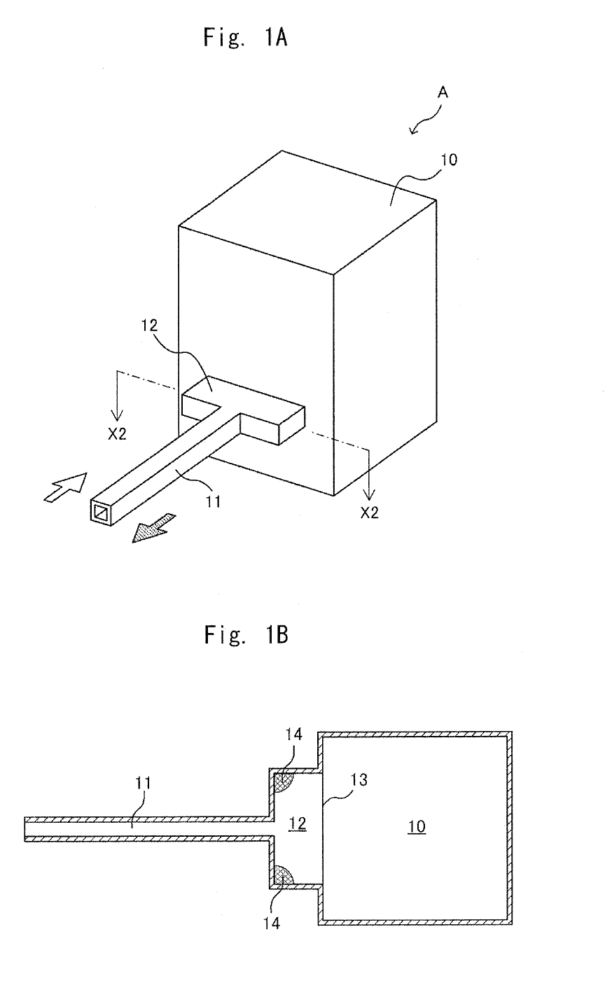

The configuration of such a laminar flow passage is explained with reference to drawings. FIG. 1 is a conceptual view showing a state where the narrow swim-up passage is communicated with the storage part by way of the laminar flow passage. Drawings referred to in the explanation made hereinafter are provided for facilitating the understanding of the present invention, and a ratio in size and the like are not always accurate. For example, a thickness, a width, a length and the like of the laminar flow passage and the narrow swim-up passage are described in an exaggerating manner with respect to the storage part. In the explanation made hereinafter, as one specific example of a shape of the opening formed in the inner wall of the storage part, a slit shape is shown. However, the present invention is not limited to such a constitution.

A sperm selection unit structure A shown in FIG. 1A includes a hollow storage part 10 which stores semen before selection therein and has a rectangular parallelepiped shape in external appearance, and a narrow swim-up passage 11 which make sperm swim up therethrough thus selecting sperm having favorable motility.

A laminar flow passage 12 which is formed of a flow passage having a wide width is interposed between the narrowed swim-up passage 11 and the storage part 10, and a downstream end potion of the laminar flow passage 12 is opened in a slit shape on an inner surface of a side wall of the storage part 10. In FIG. 1A, a blanked arrow indicates the direction of flow of a buffer solution for making sperm swim up therein, and a meshed arrow indicates the swim-up direction of sperm.

FIG. 1B is a view showing a cross section of the selection unit structure A shown in FIG. 1A taken along a line X2-X2. As can be understood also from FIG. 1B, the laminar flow passage 12 is gradually narrowed toward an upstream side of the flow of a buffer solution from the slit-shaped opening portion 13 which faces the inner surface of the side wall of the storage part 10 in an opposed manner, and the narrow swim-up passage 11 is connected to the narrow portion of the laminar flow passage 12.

With such a configuration, according to the selection unit structure A, sperm having motility which is induced by the laminar flow passage 12 can be smoothly introduced into the narrow swim-up passage 11.

According to the prior art search made by the inventors of the present invention, there exists a device provided with a micro flow passage as disclosed in WO2012/163087. This device captures sperm by balancing a swimming speed of sperm and a flow speed. Further, the device does not have a laminar flow passage. Accordingly, unlike the present invention, the device is not made based on the concept that a large amount of sperm having motility having motility exceeding predetermined motility are recovered while increasing the population of sperm as much as possible. The invention disclosed in WO2012/163087 is a device for evaluating motility of sperm by classifying sperm in accordance with motility and hence, it is safe to say that the prior art completely differs from the present invention with respect to the structure and the concept.

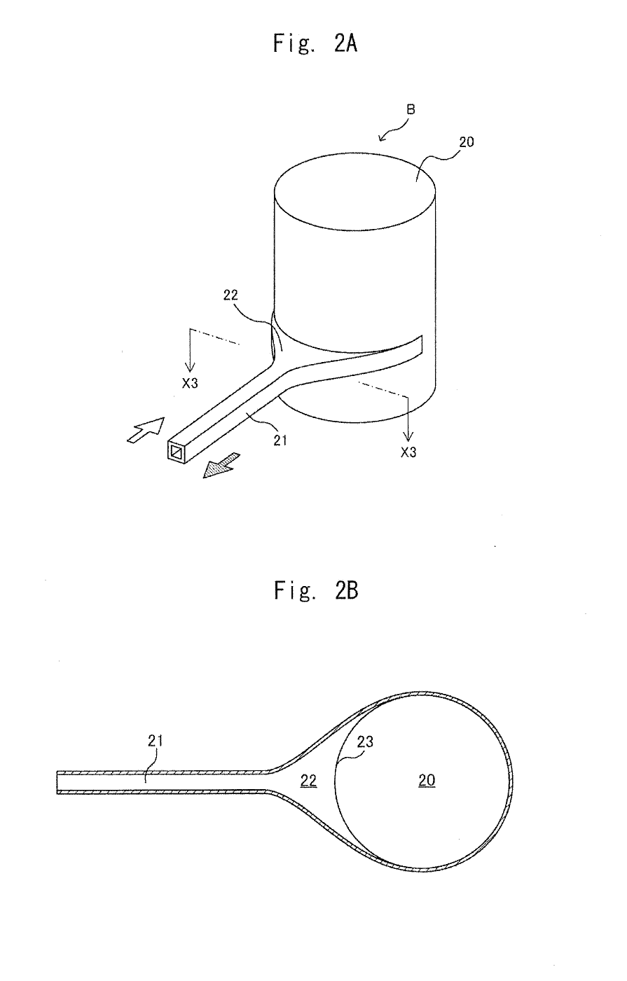

FIG. 2A shows a selection unit structure B according to a modification, and FIG. 2B shows a cross sectional view of the selection unit structure B taken along a line X3-X3 in FIG. 2A.

As shown in FIG. 2A and FIG. 2B, the selection unit structure B has the substantially same constitution and the substantially same function as the previously mentioned selection unit structure A. However, the selection unit structure B differs in structure from the selection unit structure A with respect to a point that a storage part 20 has a circular cylindrical shape, a point that a slit-shaped opening portion 23 is bent along an inner wall surface of the storage part 20, and a point that a laminar flow passage 22 is gradually narrowed while drawing a continuous curve toward an upstream side of the flow of a buffer solution as viewed in a plan view.

According to the selection unit structure B having such a constitution, it is possible to guide sperm more smoothly than the previously-mentioned selection unit structure A at the time of introducing sperm having motility induced to the laminar flow passage 22 from the storage part 20 to the narrowed swim-up passage 21.

This selection unit structure 13 is adopted by taking into account the nature of sperm that it is often the case where sperm moves along a wall at the time of swimming up and sperm have tendency of staying at corner portions. Unlike the selection unit structure. A, the selection unit structure B has no corner portions 14 indicated by meshing in FIG. 1B in the laminar flow passage 22 and hence, it is possible to prevent the stagnation of sperm thus efficiently introducing sperm into the narrow swim-up passage 21.

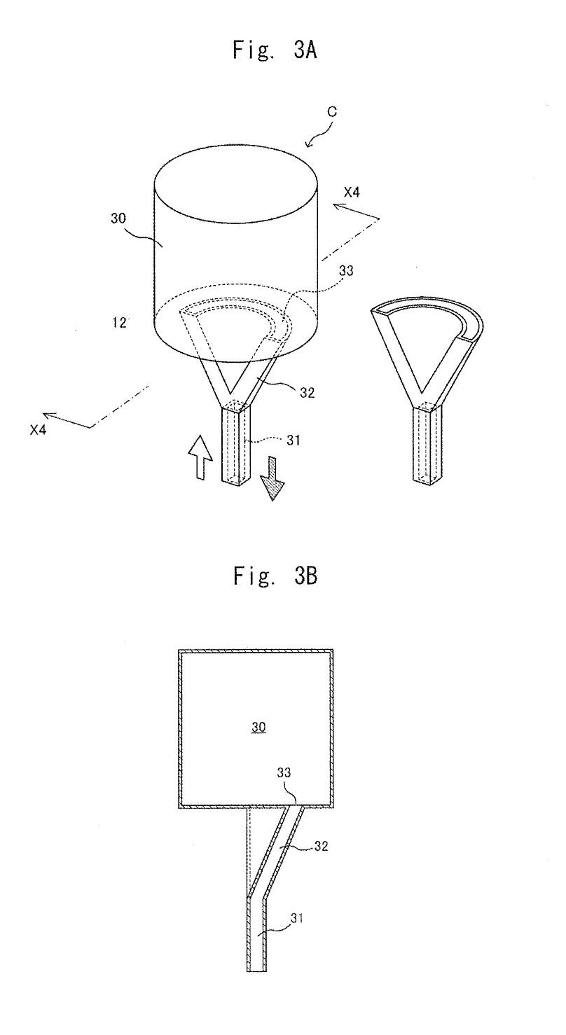

FIG. 3A and FIG. 3B are conceptual views showing a selection unit structure C according to another modification. FIG. 3A, shows the whole structure of the selection unit structure C, and FIG. 3B shows a cross-sectional view of the selection unit structure C taken along a line X4-X4 in FIG. 3A.

Although the selection unit structure C also has the substantially same function as the above-mentioned structures, as shown in FIG. 3A and FIG. 3B, the selection unit structure C differs in constitution from the above-mentioned structures with respect to a point that a laminar flow passage 32 is bent, and a point that a slit-shaped opening portion 33 of the laminar flow passage 32 is formed on a bottom wall which is one of inner wall surfaces of a storage part 30.

The selection unit structure C having such a constitution also can efficiently introduce sperm having motility to the laminar flow passage from the storage part 30 thus selecting sperm having favorable motility in the narrow swim-up passage 31.

Particularly, according to this selection unit structure C the slit-shaped opening portion 33 is formed not linearly but in a curved shape and hence, an opening area can be increased compared to a case where an opening is formed linearly and hence, sperm can be more efficiently introduced into the laminar flow passage 32.

Further, the sperm screening device according to this embodiment is characterized in that, in the sperm screening device which allows sperm recovered from an animal to swim up in a buffer solution in a laminar flow state in a predetermined zone, and includes the sperm selection unit structure according to this embodiment which recovers sperm which succeed in swimming up, the recovery passage for recovering sperm which succeed in swimming up and the buffer solution supply passage for supplying a buffer solution which flows downwardly in the swim-up passage and the recovery passage are connected to an upstream end of the swim-up passage, while a portion of the buffer solution supplied from the buffer solution supply passage is made to flow downwardly into the swim-up passage, a remaining portion of the buffer solution is made to flow downwardly into the recovery passage so as to form a recovering flow thus recovering the sperm which succeed in swimming up in the recovering flow.

With such a configuration, it is possible to recover sperm while efficiently selecting sperm having favorable motility and without applying an excessive load to the selected sperm.

It is preferable that the buffer solution supply passage have a flow passage cross-sectional area capable of supplying a flow rate of a buffer solution which enables the recovering flow to have a flow speed exceeding a swim-up ability of the sperm which succeed in swimming up through the recovering flow.

With such a configuration, it is possible to forcibly recover sperm which succeed in swimming up thus preventing the swim-up of sperm in the recovery passage.

Further, the present invention also provides a method of preparing semen for fertilization which uses the above-mentioned sperm screening device.

Particularly, the method of preparing semen for fertilization according to this embodiment includes; a sperm adding step of adding the semen before selection to the storage part; a sperm swim-up step of making sperm contained in the semen before selection added to the storage part swim up through the swim-up passage by making a buffer solution flow through the buffer solution flow passage and the recovery passage from the buffer solution supply passage at a flow speed which exceeds motility of the sperm which succeed in swimming up with respect to the recovery passage and at a flow speed which exceeds a limit flow speed by which sperm having motility but being not desired to be recovered are swimmable up and is lower than a limit flow speed by which sperm having favorable motility are swimmable up and at which a laminar flow is formed in the swim-up passage with respect to the buffer solution flow passage, and a recovery step of recovering the sperm which succeed in swimming up and are captured by the recovering flow thus preparing semen for fertilization.

Semen for fertilization which is prepared by the method of preparing semen for fertilization according to this embodiment can be used for various artificial cross fertilization such as artificial insemination (intra uterine fertilization), test tube fertilization, and in-vitro fertilization represented by intra cytoplasmic sperm injection. Particularly, it is expected that in-vitro fertilization is gradually increased in breeding cattle in the future, and semen for fertilization prepared by the method of preparing semen for fertilization according to this embodiment is extremely useful for efficiently performing cross fertilization further, semen for fertilization prepared by the method of preparing semen for fertilization according to this embodiment contains sperm having sufficient motility for fertilization with high concentration and hence, such semen have high utility as semen for artificial insemination.

While setting a predetermined timing as a threshold time, a ratio of sperm having a sex chromosome contained in the semen for fertilization may be increased by performing the sperm adding step before the threshold time elapses, or a ratio of sperm having a sex chromosome X and contained in the semen for fertilization may be increased by performing the sperm adding step after the threshold time.

For example, in a graph where the number of living sperm possessing fertility and having a sex chromosome X or a sex chromosome Y is taken on an axis of ordinates and time is taken on an axis of abscissas, the threshold time may be set as time corresponding to a valley between a peak of the number of living sperm having a sex chromosome X and acquiring fertility and a peak of the number of living sperm having a sex chromosome Y and acquiring fertility.

For example, the threshold time may be set as a timing at which sperm contained in semen before selection and having a sex chromosome X acquires fertility. These threshold times may be suitably decided in conformity with a state of sperm or the like

In the method of preparing semen for fertilization according to this embodiment, while setting a predetermined timing as a threshold time, a ratio of sperm having a sex chromosome Y contained in the semen for fertilization may be increased by performing the sperm adding step before the threshold time elapses, or a ratio of sperm having a sex chromosome X and contained in the semen for fertilization may be increased by performing the sperm adding step after the threshold time.

Semen for fertilization acquired by such a method contains a larger amount of sperm having a sex chromosome X than an amount of sperm having a sex chromosome Y and hence, such semen for fertilization is advantageous for acquiring femininity.

Further, the method of preparing semen for fertilization according to this embodiment may be configured such that, in a sperm preservation step in which the semen before selection is preserved before the sperm adding step is performed, among sperm in the semen before selection, motility of sperm having either one of sex chromosomes out of the sperm having the sex chromosome X and the sperm having the sex chromosome Y is enhanced and hence, the swim-up of sperm having either one of the sex chromosomes in the swim-up passage is accelerated in the sperm swim-up step whereby a ratio of sperm having either one of sex chromosomes and contained in the semen for fertilization acquired by the recovery step is increased.

That is, by enhancing motility of sperm having either one of a sex chromosome X and a sex chromosome Y out of sperm contained in semen before selection which is used in the sperm adding step, it is possible to make the sperm swim up efficiently thus preparing semen for fertilization which is advantageous for acquiring either one of a male individual and a female individual.

Further, the method of preparing semen for fertilization according to this embodiment may be configured such that, in the sperm preservation step in which the semen before selection is preserved before the sperm adding step is performed, among sperm in the semen before selection, motility of sperm having either one of sex chromosomes out of sperm having sex chromosome X and sperm having sex chromosome Y is lowered so that the swim-up of sperm having either one of sex chromosomes in the swim-up passage is suppressed in the sperm swim-up step whereby a ratio of sperm having either one of sex chromosomes and contained in the semen for fertilization acquired in the recovery step is increased.

That is, by lowering motility of sperm having either one of a sex chromosome X and a sex chromosome V out of sperm contained in semen before selection which is used in the sperm adding step, the sperm having the other sex chromosome are allowed to swim up preferentially thus preparing semen for fertilization which is advantageous for acquiring either one of male individuals and female individuals.

The method of preparing semen for fertilization according to this embodiment may be configured such that a preservation time in the sperm preservation step in which the semen before selection are preserved before the sperm adding step is performed is prolonged so that a ratio of sperm having sex chromosome X and contained in the semen for fertilization may be increased.

Sperm having an X chromosome acquire fertility later than sperm having a V chromosome and hence, by prolonging preservation time of the sperm in a sperm preservation step, the number of sperm which acquire fertility and have an X chromosome in semen for fertilization can be increased thus preparing semen for fertilization which is advantageous for acquiring male individuals. Although the preservation time at this stage of operation is not particularly limited, for example, by prolonging the preservation time more than the threshold time, it is possible to prepare semen for fertilization capable of acquiring male individuals more advantageously.

The method of preparing semen for fertilization according to this embodiment may be configured such that, in the sperm preservation step in which the semen before selection is preserved before the sperm adding step is performed, a phenoxazine compound is present in the semen before selection so that a ratio of sperm having sex chromosome Y contained in the semen for fertilization is increased.

In the method of preparing semen for fertilization according to this embodiment, it is preferable that the sperm preservation step be performed under the condition that an electron accepting substance is not present. Here, “under the condition that an electron, accepting substance is not present” means “under the condition that intentionally added electron accepting substance is not present”, and does not mean “under the condition that electron accepting substance which are originally present in sperm and semen are also not present”.

By performing the sperm preservation step under such a condition, it is possible to prepare semen for fertilization which contains a large amount of sperm having a sex chromosome V and is advantageous for acquiring male individuals.

The method of preparing semen for fertilization according to this embodiment may be configured such that, in the sperm preservation step in which the semen before selection is preserved before the sperm adding step is performed, a phenoxazine compound and an electron accepting substance are present in the semen before selection so that a ratio of sperm having sex chromosome X and contained in the semen for fertilization may be increased.

By performing the sperm preservation step under such a condition, it is possible to prepare semen for fertilization which contains a large amount of sperm having a sex chromosome X and is advantageous for acquiring female individuals.

Further, the electron accepting substance may be at least one selected from a group consisting of flavins, phenazines, and NAD(P)H oxidoreductase. For example, when NAD(P)H oxidoreductase is used as the electron accepting substance, it is preferable that the concentration of NAD(P)H oxidoreductase in semen before selection used in the sperm preservation step he set to approximately 0.1 to 10 U/ml. When the concentration of NAD(P)H oxidoreductase is less than 0.1 U/ml, it is difficult for the electron accepting substance to exert sufficient influence on sperm. Further, even when the concentration of NAD(P)H oxidoreductase exceeds 10 U/ml, it is difficult for the electron accepting substance to acquire a further advantageous effect, and there arises a concern about toxicity to the sperm to the contrary. By setting the concentration of an electron accepting substance to a value which falls within a range from 10 U/ml inclusive, it is possible to exert sufficient influence on sperm while suppressing toxicity to sperm.

Further, in the sperm preservation step, hexose-6-phosphate may be further added to the semen before selection when necessary.

Although the above-mentioned phenoxazine compound, hexose-6-phosphate and electron accepting substance are not particularly limited, to be more specific, it is possible to use following substances.

That is, a phenoxazine compound may be one or a mixture of two or more selected from a group consisting of brilliant cresyl blue, nile blue, basic blue and meldola blue, for example, it is preferable that the concentration of phenoxazine compound in the semen before selection used in the sperm preservation step he set to an approximately 1 to 100 μM. When the concentration of phenoxazine compound is less than 1 μM, it is difficult for the phenoxazine compound to exert sufficient influence on the sperm. On the other hand, even when the concentration of phenoxazine compound exceeds 100 μM, it is difficult for the phenoxazine compound to further increase such an advantageous effect and there is concern about toxicity to the sperm to the contrary. By setting the concentration of the phenoxazine compound to a value which falls within a range from 1 μM to 100 μM inclusive, a phenoxazine compound can exert sufficient influence on the sperm while suppressing toxicity to the sperm.

The hexose-6-phosphate may he one or a mixture of two or more selected from a group consisting of D-glucose 6-phosphate, fructose-6-phosphate, mannose-6-phosphate and galactose-6-phosphate, for example. It is preferable that the concentration of hexose-6-phosphate in the semen before selection used in the sperm preservation step be set to an approximately 0.1 to 100 mM. When the concentration of hexose-6-phosphate is less than 0.1 mM, it is difficult for hexose-6-phosphate to exert sufficient influence on the sperm. Further, even when the concentration of hexose-6-phosphate exceeds 100 mM, it is difficult for hexose-6-phosphate to acquire a further advantageous effect and there arises a concern that hexose-6-phosphate impedes the advantageous effect to the contrary. By setting the concentration of the hexose-6-phosphate to a value which fails within a range from 0.1 μM to 100 μM inclusive, it is possible to exert sufficient influence on the sperm while suppressing toxicity to the sperm.

Hereinafter, the sperm selection unit structure, the sperm screening device, and the method of preparing semen for fertilization using the sperm screening device according to this embodiment are explained in more detail with reference to drawings. In the explanation made hereinafter, as one specific example of semen for fertilization, semen for artificial insemination (intra uterine fertilization) is referred. However, it is needless to say that semen for fertilization is not limited to semen for artificial insemination, and it is also applicable as semen used for the test tube fertilization and the infra cytoplasmic sperm injection.

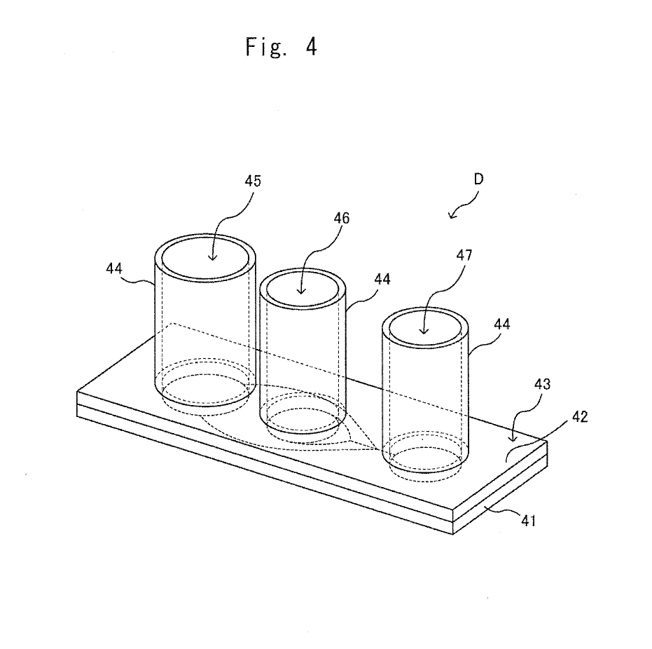

FIG. 4 is an explanatory view showing the constitution of a sperm screening device D according to this embodiment. As shown in FIG. 4, the sperm screening device D is configured such that three cylindrical bodies 44 are placed on a body part 43 which is formed by laminating a bottom portion forming plate 41 and a chip body 42 in an overlapping manner. Further, a buffer solution storage part 45, a storage part 46 for storing semen before selection, and a recovery part 47 are formed in the inside of cylindrical bodies 44 respectively.

Then, a predetermined amount of buffer solution is filled into the buffer solution storage part 45, a predetermined amount of semen before selection is filled into the storage part. 46 for storing semen before selection, and a predetermined amount of buffer solution is also filled into the recovery part 47. With such a configuration, it is possible to select sperm having favorable motility from sperm contained in the semen before selection stored in the storage part 46 for storing semen before selection due to an action of a micro fluid and to recover such sperm in the recovery part 47 together with a buffer solution.

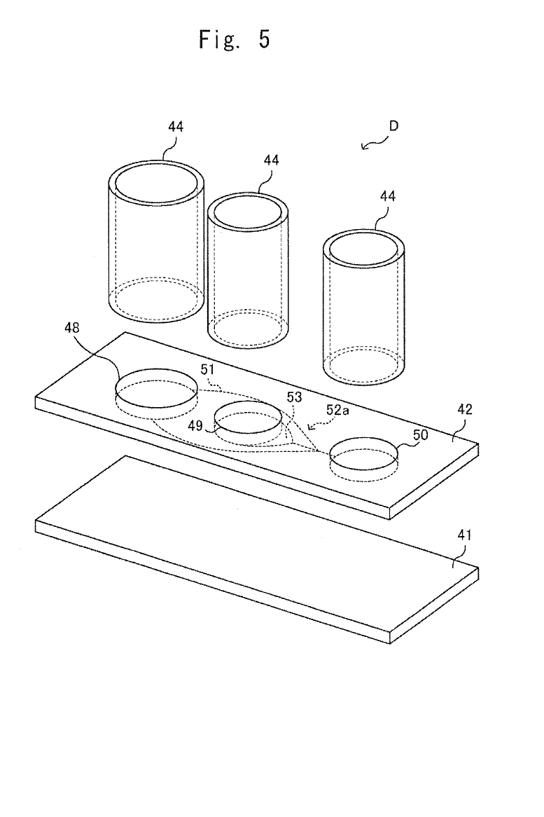

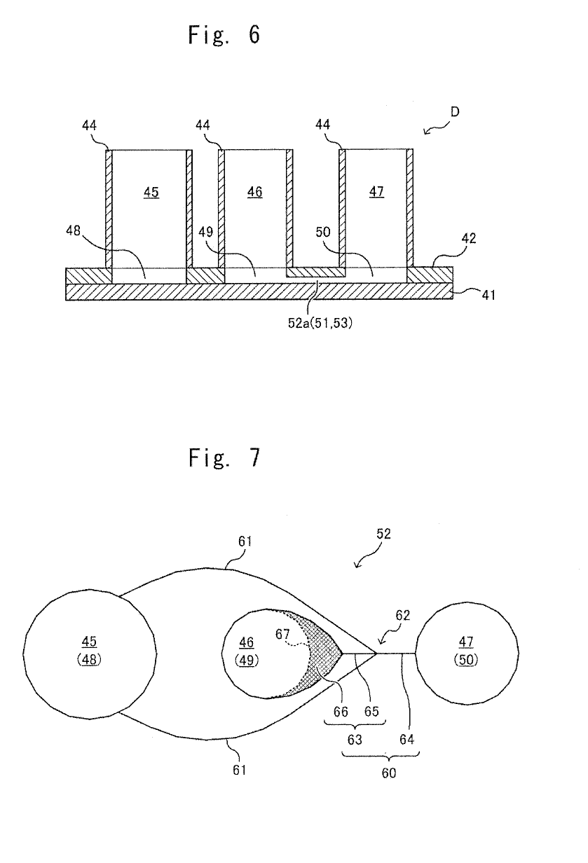

As shown in FIG. 5 which is an exploded perspective view and FIG. 6 which is a cross-sectional view, the bottom portion forming plate 41 which constitutes the body part 43 is a substrate on which the chip body 42 is placed. The bottom portion forming plate 41 plays a role of a member which forms bottom portions of the buffer solution storage part 45, the storage part 46 for storing semen before selection and the recovery part 47, and forms a bottom portion of a flow passage pattern 52a described later which is formed on a back surface of the chip body 42. The bottom portion forming plate 41 can be made of glass or a resin (acrylic or PDMS), for example.

The chip body 42 which constitutes the body part 43 is a plate-like member made of a resin, and in the embodiment, the body part 43 is made of polymethyl siloxane (PDMS).

Three holes are formed in the chip body 42. These three holes form a buffer solution hole portion 48 which constitutes a portion of the previously mentioned buffer solution storage part 45, a storage part hole portion 49 which constitutes a portion of the storage part 46 for storing semen before selection, and a recovery part hole portion 50 which constitutes a portion of the recovery part 47 respectively.

The flow passage pattern 52a is formed on a back surface of the chip body 42 by microfabrication. The flow passage pattern 52a is provided for forming the micro flow passage 52 by laminating the bottom portion forming plate 41 and the chip body 42 to each other in an overlapping manner. The flow passage pattern 52a is formed by forming a plurality of groove portions 51 which make the buffer solution hole portion 48, the storage part hole portion 49 and the recovery part hole portion 50 communicate with each other and by forming a laminar flow passage forming recessed portion 53 for forming the previously mentioned laminar flow passage.

The respective cylindrical bodies 44 are arranged on peripheries of the buffer solution hole portion 48, the storage part hole portion 49, and the recovery hole potion 50 respectively thus functioning as wall bodies for increasing storage capacities of the buffer solution storage part 45, the storage part 46 for storing semen before selection, and the recovery part 47. The respective cylindrical bodies 44 have outer diameters larger than diameters of hole portions thereof respectively, and are arranged and fixed to peripheries around the respective holes watertightly.

Next, the constitution of the micro flow passage 52 which is formed by laminating the bottom portion forming plate 41 and the chip body 42 to each other in an overlapping, manner is explained with reference to FIG. 7.

As shown in FIG. 7, the micro flow passage 52 is constituted of a storage part/recovery part communicating flow passage 60 which communicably connects the storage part hole portion 49 constituting a portion of the wall surface of the storage part 46 for storing semen before selection and the recovery part hole potion 50 to each other, and two buffer solution supply passages 61 which are connected to an intermediate portion of the storage part/recovery part communicating flow passage 60 from the buffer solution hole portion 48. Further, a portion where the storage part/recovery part communicating flow passage 60 and the buffer solution supply passages 61 intersect with each other is formed as a merging portion 62.

The storage part/recovery part communicating flow passage 60 is constituted of a swim-up passage 63 which is formed in the direction toward the storage part 46 for storing semen before selection from the merging portion 62, and a recover passage 64 which is formed in the direction toward the recovery part 47 from the merging portion 62.

The buffer solution supply passage 61 is a flow passage for supplying a buffer solution stored in the buffer solution storage part 45 to the merging portion 62. The buffer solution storage part 45 and the buffer solution supply passage 61 function as the previously-mentioned buffer solution supply means.

A portion of a buffer solution which is supplied to the merging portion 62 by the buffer solution supply passage 61 flows to the swim-up passage 63, and a remaining portion of the buffer solution flows to the recovery passage 64. In the explanation made hereinafter, the flow of a buffer solution which flows into the swim-up passage 63 and flows downwardly is referred to as a swim-up flow, and the flow of a buffer solution which flows into the recovery passage 64 and flows downwardly is referred to as a recovery flow.

The swim-up passage 63 is a passage for inducing sperm from semen before selection stored in the storage part 46 for storing semen before selection and for selecting sperm having favorable motility, and the swim-up passage 63 is constituted of a narrow swim-up passage 65 and a laminar flow passage 66.

The narrow swim-up passage 63 is a passage for preventing deformed sperm and sperm having inferior motility to an extent that the sperm are not favorable to perform the artificial insemination from reaching the merging portion 62 by making such sperm drop out in the swim-up flow while making the sperm having favorable motility swim up in the swim-up flow generated by the buffer solution branched in the merging portion 62.

As has been explained with reference to FIG. 1 to FIG. 3, the laminar flow passage 66 is a portion which functions as an inducing means for efficiently introducing sperm in the storage part 46 for storing semen before selection to the narrow swim-up passage 65, and is a portion indicated by meshing in FIG. 7. A broken line shown in a downstream-side end portion (a downstream-side end portion of the swim-up passage 63) of the laminar flow passage 66 indicates a slit-shaped opening portion 67 which opens on a side wall portion of the storage part 46 for storing semen before selection.

In this manner, in the micro flow passage 52, the sperm selection unit structure according to this embodiment is constituted by the narrow swim-up passage 65, the laminar flow passage 66, and the slit-shaped opening portion 67.

The recovery passage 64 is a flow passage for recovering sperm which swim up in the narrow swim-up passage 65 against the swim-up flow and reach the merging portion 62, and is a portion for making the sperm reaching the merging portion 62 flow into the recovery portion 47 together with the recovery flow.

According to the micro flow passage 52 having such a constitution, the sperm adding step is performed where semen before selection is added to the storage part 46 for storing semen before selection, and sperm having favorable motility are efficiently selected from the semen before selection stored in the storage part 46 for storing semen before selection, that is, from the large population of sperm, by the slit-shaped opening portion 67, the laminar flow passage 66, and the narrow swim-up passage 65, and are recovered by the recovery part 47 together with the recovery flow.

Particularly, in the micro flow passage 52 of the sperm screening device D according to this embodiment, the buffer solution supply passage 61 is formed such that a flow speed of a buffer solution in the swim-up flow exceeds a limit flow speed by which sperm having motility but being not desired to be recovered are swimmable up and is below a limit flow speed by which sperm having favorable motility are swimmable up, and is set to a flow speed at which the buffer solution flow becomes a laminar flow in the swim-up passage 63 (narrow swim-up passage 65 and the laminar flow passage 66).

In the micro flow passage 52 of the sperm screening device D according to this embodiment, the buffer solution supply passage 61 is formed such that a flow speed of a buffer solution in the recovery flow becomes a flow speed exceeding motility of sperm which reach the merging portion 62 (succeed in swimming up).

That is, the buffer solution supply passages 61 are configured such that two buffer solution supply passages 61 provide a flow passage cross-sectional area capable of supplying a buffer solution to the merging portion 62 at a flow rate which can generate a swim-up flow and a recovery flow which satisfy the above-mentioned conditions.

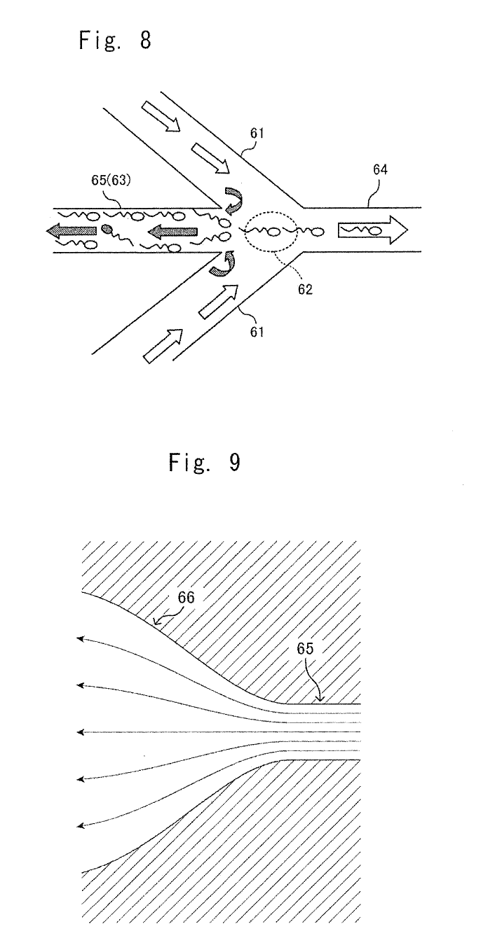

Accordingly, as shown in FIG. 8, when a buffer solution supplied by two buffer solution supply passages 61 reaches the merging portion 62, a portion of the buffer solution flows into the swim-up passage 63 (narrow swim-up passage 65) as indicated by a meshed arrow so that a laminar-flow-like swim-up flow through which sperm having favorable motility can swim up and though which sperm having inferior motility can hardly swim up is formed.

A remaining portion of the buffer solution supplied by the buffer solution supply passage 61 flows into the recovery passage 64 as indicated by a blanked arrow so that a recovery flow for recovering sperm which can hardly swim up although having favorable motility is formed.

The swim-up flow flows downwardly in the narrow swim-up passage 65 and reaches the laminar flow passage 66. Then, the laminar flow passage 66 is formed of a flow passage having a wide width where the swim-up flow flows therethrough downwardly as a slow flow while maintaining a laminar flow state and hence, as shown in FIG. 9, the flow which is slower than the swim-up flow flowing in the narrow swim-up passage 65 and maintains a laminar flow state is formed.

Accordingly, the wide laminar swim-up flow where a flow speed is lowered can be made to flow out from the slit-shaped opening portion 67, while it is possible to keep sperm having no motility and motes away from the opening, it is possible to induce a large amount of sperm into the laminar flow passage 66 by allowing the sperm to generate the property of rheotaxis while ensuring a large frontage width with respect to sperm having motility.

Further, a buffer solution is made to flow through the swim-up passage 63 (buffer solution flow passage) and the recovery passage 64 from the buffer solution supply passages 61 at a flow speed which exceeds motility of sperm which succeed in swimming up in the recovery passage 64 and at a flow speed which exceeds a limit flow speed by which sperm having motility but being not desired to be recovered are swimmable up and is below a limit flow speed by which sperm having favorable motility are swimmable up and where the flow of the buffer solution becomes a laminar flow in the swim-up passage 63 (buffer solution flow passage), and the sperm swim-up step where sperm contained in the storage part 46 for storing semen before selection swim up the swim-no passage 63 is performed.

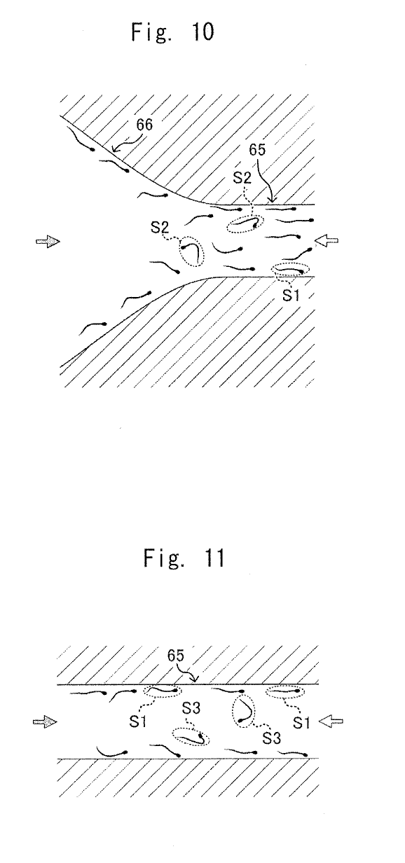

The laminar flow passage 66 is formed into a gradually narrowed shape toward an upstream side of the swim-up flow and hence, as shown in FIG. 10, sperm S induced by the laminar flow passage 66 is gradually recovered to an inlet portion of the narrow swim-up passage 65 so that sperm S1 can be efficiently introduced to the narrow swim-up passage 65.

A flow speed of the buffer solution is gradually (continuously) increased in the vicinity of the inlet portion of the narrow swim-up passage 65 and hence, the swim-up of sperm which has motility but cannot move normally such as deformed sperm is obstructed.

Further, sperm 52 which cannot swim up against a flow speed of the swim-up flow in the narrow swim-up passage 65 also drop out.

In the narrow swim-up passage 65, the swim-up flow flows at a flow speed at which sperm S3 having inferior motility cannot swim up and sperm S1 having favorable motility can swim up and hence, as shown in FIG. 11, the sperm S3 having inferior motility drop out, and the sperm S1 having favorable motility are selected.

Then, when the sperm S1 having favorable motility swim up to the upstream-side end portion of the narrow swim-up passage 65 and reach the merging portion 62, due to the flow of the buffer solution directed toward the recovery passage 64 from the buffer solution supply passages 61, the sperm S1 are drawn to the inlet portion of the recovery passage 64 and are fed to the recovery part 47 due to the recovery flow. By performing the recovering step where swim-up sperm which are caught by the recovery flow are recovered, semen for artificial insemination is prepared. That is, a solution stored in the recovery part 47 which contains fed sperm contains sperm having favorable motility at an unprecedentedly high concentration and hence, such a solution can be used as semen for artificial insemination.

In this manner, the sperm screening device D according to this embodiment includes the sperm selection unit structure according to this embodiment and hence, it is possible to efficiently separate sperm having favorable motility.

[Sperm Screening Test]

Next, the explanation is made with respect to the sperm screening test performed by, the sperm screening device configured by using chip bodies having various flow passage patterns. This test is also a test for preparing semen for fertilization. To be more specific, this test is also a test for preparing semen for artificial insemination.

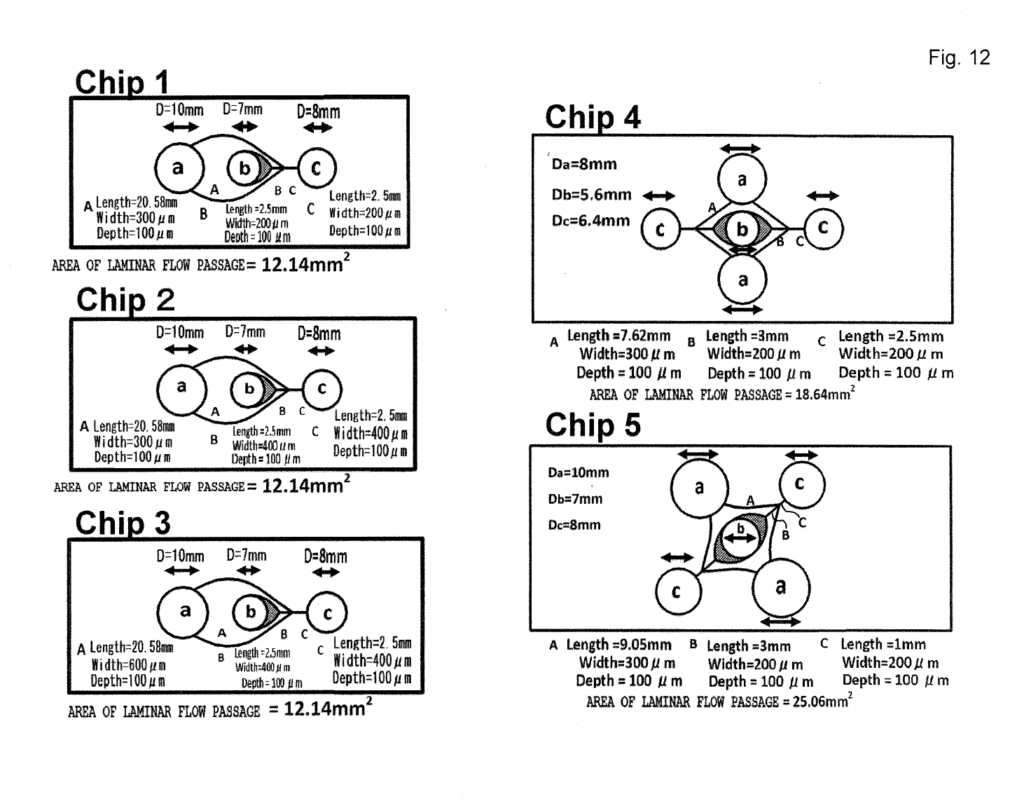

In this test, the test was carried out by replacing a portion which corresponds to the chip body 42 of the sperm screening device D with various chip bodies shown in FIG. 12, and the number of recovered sperm, a ratio of sperm having motility, linear velocity, curvilinear velocity, and linearity were acquired.

The chip 1 shown in FIG. 12 has the substantially same constitution as the previously-mentioned micro flow passage 52 as viewed in a plan view. An inner diameter of a buffer solution storage part indicated by symbol “a” is 10 mm, an inner diameter of a storage part for storing semen before selection indicated by symbol “b” is 7 mm, an inner diameter of a recovery part indicated by symbol “c” is 8 mm, a length of a buffer solution supply passage indicated by symbol “A” is 20.58 mm, a width of the buffer solution supply passage A is 300 μm, a depth of the buffer solution supply passage A is 100 nm, a length of a narrow swim-up passage indicated by symbol “B” is 2.5 mm, a width of the narrow swim-up passage B is 200 μm, a depth of the narrow swim-up passage B is 100 μm, a length of a recovery passage 64 indicated by symbol “C” is 2.5 mm, a width of the recovery passage 64 is 200 μm, a depth of the recovery passage 64 is 100 μm, a depth of a meshed laminar flow passage is 100 μm, and an area of the laminar flow passage as viewed in a plan view is 12.14 mm².

The chip 2 shown in FIG. 12 has the substantially same flow passage constitution as the previously-mentioned micro flow passage 52 as viewed in a plan view in the same manner as the previously-mentioned chip 1. However, the width of the narrow swim-up passage and the width of the recovery passage are set to 400 nm while not changing a flow rate of a swim-up flow and a flow rate of a recovery flow thus providing the flow whose flow speed is halved.

The chip 3 shown in FIG. 12 has the substantially same flow passage constitution as the previously-mentioned micro flow passage 52 as viewed in a plan view in the same manner as the previously-mentioned chip 2. However, a width of a buffer solution supply passage is also set to 600 μm. That is, the chip 3 is configured to provide the flow having a flow speed twice as large as a flow speed of the flow in the chip 1 and a flow rate of the flow twice as large as a flow rate of the flow in the chip 1.

The chip 4 is configured such that two swim-up passages are provided to one storage part for storing semen before selection. To be more specific, an inner diameter of two buffer solution storage parts indicated by symbol “a” is 8 mm, an inner diameter of the storage part for storing semen before selection indicated by symbol “h” is 5.6 mm, an inner diameter of two recovery parts indicated by symbol “c” is 6.4 mm, a length of four buffer solution supply passages indicated by symbol “A” is 7.62 mm, a width of four buffer solution supply passages A is 300 μm, a depth of four buffer solution supply passages A is 100 μm, a length of two narrow swim-up passages indicated by symbol B is 3 mm, a width of two narrow swim-up passages B is 2003 μm, a depth of two narrow swim-up passages B is 100 μm, a length of two recovery passages 64 indicated by symbol “C” is 2.5 mm, a width of two recovery passages 64 is 200 μm, a depth of two recovery passages 64 is 100 μm, a depth of two laminar flow passages is 100 μm, and an area of each laminar flow passage as viewed in a plan view is 18.64 mm².

In the same manner as the chip 4, a chip 5 is also configured such that two swim-up passages are provided to one storage part for storing semen before selection. To be more specific, an inner diameter of two buffer solution storage parts indicated by symbol “a” is 10 mm, an inner diameter of the storage part for storing semen before selection indicated by symbol “h” 7 mm, an inner diameter of two recovery parts indicated by symbol “c” is 8 mm, a length of four buffer solution supply passages indicated by symbol “A” is 9.05 mm, a width of four buffer solution supply passages A is 300 μm, a depth of four buffer solution supply passages A is 100 μm, a length of two narrow swim-up passages indicated by symbol “B” is 3 mm, a width of two narrow swim-up passages B is 200 μm, a depth of two narrow swim-up passages B is 100 μm a length of two recovery passages 64 indicated by symbol “C” is 1 mm, a width of two recovery passages 64 is 200 μm, a depth of two recovery passages 64 is 100 μm, a depth of two laminar flow passages is 100 μm, and an area of each laminar flow passage as viewed in a plan view is 25.06 mm².

The sperm screening devices are configured by using these chips 1 to 5, and a sperm screening test was performed by adding a buffer solution and thawed frozen semen which forms semen before selection to the respective chips.

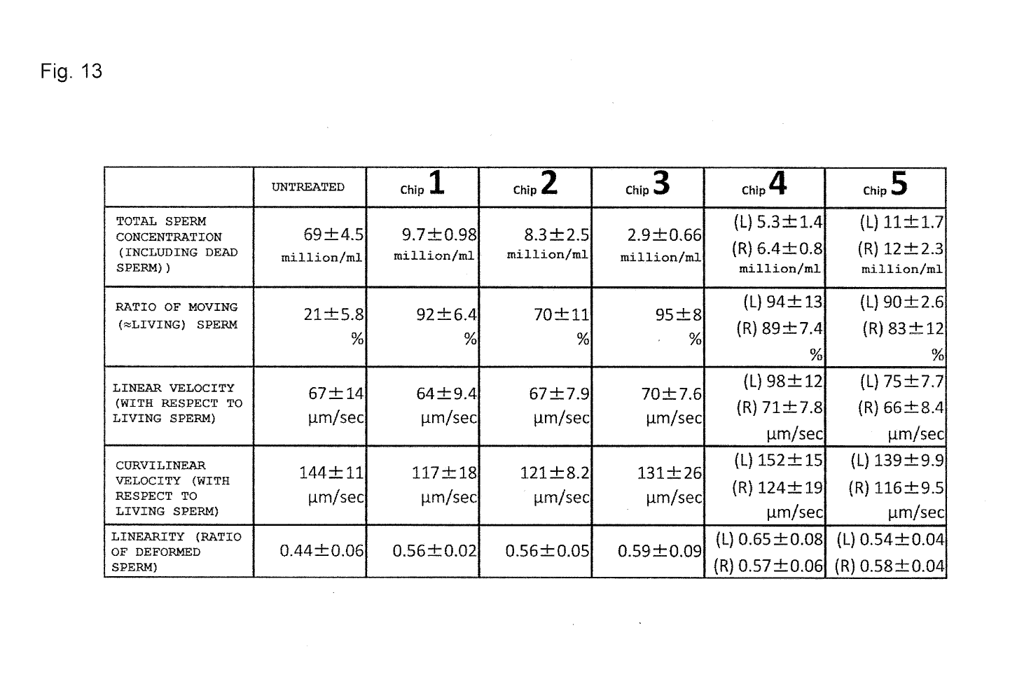

In the thawed frozen semen used in the tests, a total sperm concentration was 69±4.5 million/ml, a ratio of moving sperm was 21±5.8%, a linear velocity was 67±14 μm/sec, a curvilinear velocity was 144±11 μm/sec, and a linearity was 0.44±0.06. Further, a screening time is set to 30 minutes. Test results are shown in FIG. 13.

As can be understood from FIG. 13, all chips 1 to 5 could efficiently separate sperm at least at a concentration of several millions/ml per 30 minutes which is remarkably higher than a conventional level of several tens/ml per minute. That is, semen suitable for an artificial insemination were prepared.

To focus on a ratio of moving (living) sperm, it is understood that while a ratio of living sperm was 21±5.8% in thawed frozen semen (untreated) used in the test, a ratio of living sperm in the recovered sperm was increased to 70% or more in all chips 1 to 5.

The test result also indicates that the linearity was also enhanced so that deformed sperm were also removed extremely efficiently.

In this manner, the test result indicates that the sperm screening device according to this embodiment could recover sperm having favorable motility extremely efficiently compared to the conventional sperm screening which makes sperm swim up using a buffer solution flow passage as a swim-up passage.

With the use of the sperm screening device according to this embodiment, it is also possible to bias a female/male conception probability in the artificial insemination toward a desired sex.

That is, according to the method of preparing semen for artificial insemination of this embodiment, it is possible to prepare semen for artificial insemination where a ratio of sperm having a sex chromosome X (hereinafter also referred to as X sperm) or sperm having a sex chromosome Y (hereinafter also referred to as Y sperm) is arbitrarily increased.

Such a method of preparing semen for artificial insemination is further explained hereinafter.

Conventionally, the production of livestock, for example, the production of cattle having a desired sex has been attempted by livestock farmers.

The fact is, however, that the sex selection heavily depends on experience and intuition of the livestock farmers and has not been established as a generalized technique.

Particularly, with respect to semen (semen before selection) which is prepared by mixing two semen recovered from the same male individuals at different timings, fertility capacitation timings are dispersed and hence, it is difficult to say that good results can be obtained.

On the other hand, with the use of the sperm screening device according to this embodiment, it is possible to realize the preparation of semen for artificial insemination where a ratio of X sperm or Y sperm is larger than a ratio of X sperm or sperm in the semen before selection. Eventually, it is possible to enhance the conception probability of livestock having a desired sex.

To be more specific, the method of preparing semen for artificial insemination is the method of preparing semen for artificial insemination which uses the previously-mentioned sperm screening device, wherein the sperm screening device includes: the storage part which stores semen containing sperm recovered from an animal therein; the buffer solution flow passage which is communicated with the storage part and makes a buffer solution flow therethrough in the direction toward the storage part as a laminar flow; and the swim-up passage which is communicated with the storage part so as to make sperm swim up in the buffer solution flow passage from the storage part. The downstream portion of the swim-up passage is formed of a flow passage having a wide with where a buffer solution flows as a slow flow while maintaining a laminar flow state of the buffer solution, and the downstream end portion of the flow passage is opened in a state where the downstream end portion faces the wall surface of the storage part. The recovery passage for recovering sperm which succeed in swimming up and the buffer solution supply passage for supplying a buffer solution which flows downwardly in the swim-up passage and the recovery passage are connected to the upstream end of the swim-up passage. While a portion of the buffer solution supplied from the buffer solution supply passage is made to flow downwardly into the swim-up passage, a remaining portion of the buffer solution is made to flow downwardly into the recovery passage so as to form a recovering flow thus collecting the sperm which succeed in swimming up in the recovering, flow. That is, the method of preparing semen for artificial insemination is based on the method of preparing semen for artificial insemination which includes: a sperm adding step of adding the semen before selection to the storage part; a sperm swim-up step of making sperm added to the storage part swim up through the swim-up passage by making a buffer solution flow through the buffer solution flow passage and the recovery passage from the buffer solution supply passage at a flow speed which exceeds motility of the sperm which succeed in swimming up with respect to the recovery passage and at a flow speed which exceeds a limit flow speed by which sperm having motility but being not desired to be recovered are swimmable up and is Lower than a limit flow speed by which sperm having favorable motility are swimmable up and at which a laminar flow is formed in the swim-up passage with respect to the buffer solution flow passage, and a recovery step of recovering the sperm which succeed in swimming up and are captured by the recovering flow.

Characteristically, while setting a timing at which sperm contained in the semen before selection and having a sex chromosome X acquires fertility as a threshold time, a ratio of sperm having a sex chromosome contained in the semen for artificial fertilization is increased by performing the sperm adding step before the threshold time elapses, or a ratio of sperm having a sex chromosome X and contained in the semen for artificial fertilization is increased by performing the sperm adding step after the threshold time.

To further explain the method of preparing semen for artificial insemination by taking cattle as an example of livestock, among male cattle, there are individuals which secrete semen containing X sperm which acquire fertility at a relatively early stage, and there are individuals which secrete semen containing X sperm which acquire fertility at a delayed time. Further, even in the same individual, a state of sperm in semen changes depending on a physical condition at a certain point of time the season and the like. Accordingly, the above-mentioned threshold time is decided individually or suitably decided corresponding to a state of each individual. The threshold time can be decided by performing a preliminary examination for every individual with respect to a time which is required until X sperm starts the acquisition of fertility.

It is also safe to say that the method of preparing semen for artificial insemination explained in this embodiment is a method of preparing semen for artificial insemination where a preservation time in the sperm preservation step in which the semen before selection is preserved before the previously-mentioned sperm adding step is performed is prolonged so that a ratio of sperm having sex chromosome X and contained in the semen for artificial insemination is increased.

Hereinafter, further experimental examples with respect to the method of preparing semen for artificial insemination according to this embodiment are described.

### Experimental Example 1

After thawing frozen semen of “Shinnosuke; (Japanese black cattle)” purchased from JA SAGA at a temperature of 37° C., seminal plasma (having an action of suppressing capacitation), a cryoprotectant and the like were removed by the centrifugal separation.

Thereafter, a threshold time was set to 10 hours after a point of time at which the semen is thawed based on the result of the preliminary examination performed in advance. The sperm preservation step where the semen is preserved for 9 hours or 15 hours at 37° C. was performed and, thereafter, sperm having high motility were recovered by using the sperm screening device according to this embodiment.

Subsequently, a ratio between Y sperm and X sperm in captured sperm was examined using a real time PCR method. The result of the examination is shown in Table 1.

TABLE 1

| | X | Y |
|---|---|---|
| frozen semen immediately after being thawed | 52.00% | 48.00% |
| (semen before selection) | | |
| semen for artificial insemination acquired by | 42.70% | 57.30% |
| being preserved for 9 hours after being thawed | | |
| and by being supplied to screening device | | |
| semen for artificial insemination acquired by | 59.40% | 40.60% |
| being preserved or 15 hours after being thawed | | |
| and by being supplied to screening device | | |

### Experimental Example 2

In the same manner as the previously-mentioned experimental example 1, the examination was made with respect to “Shichiho-jin; (Japanese black cattle)” purchased from JA SAGA. The result of the examination is shown in Table 2.

TABLE 2

| | X | Y |
|---|---|---|
| frozen semen immediately after being thawed | 49.00% | 51.00% |
| (semen before selection) | | |
| semen for artificial insemination acquired by | 38.30% | 61.70% |
| being preserved for 3 hours after being thawed | | |
| and by being supplied to screening device | | |
| semen for artificial insemination acquired by | 38.10% | 61.90% |
| being preserved for 6 hours after being thawed | | |
| and by being supplied to screening device | | |
| semen for artificial insemination acquired by | 40.00% | 60.00% |
| being preserved for 15 hours after being thawed | | |
| and by being supplied to screening device | | |

As can be understood from Table 1 and Table 2, in all experimental examples, provided that the preservation time is less than 10 hours which is the threshold time, a ratio of Y sperm could be increased more than a ratio of Y sperm in the semen before selection.

On the other hand, when the preservation time is more than 10 hours which is the threshold time, a ratio of X sperm could be increased more than a ratio of X sperm in the semen before selection and a ratio of X sperm in semen selected before the threshold time elapses (see Table 1), or could be increased more than a ratio of X sperm in the semen selected before the threshold time elapses (see Table 2).

From the results of the examinations, it is found that the method of preparing semen for fertilization using the sperm screening device according to this embodiment can realize the preparation of semen for fertilization where a ratio of Y sperm is increased more than a ratio of Y sperm in the semen before selection, and the preparation of semen for fertilization where a ratio of X sperm is increased more than a ratio of X sperm in the semen before selection and a ratio of X sperm in the semen selected before the threshold time elapses. Eventually, it is possible to enhance the conception probability of livestock having a desired sex.

That is, it is also considered that the method of preparing semen for fertilization according to this embodiment described above is a method of making the maturation periods of sperm suitable for fertilization uniform to some extent by screening the sperm in the semen before selection depending on motility and, further, increasing a ratio of sperm having a desired sex chromosome while adjusting a fertilization acquisition timing.

Accordingly, it is possible to prepare semen for fertilization where a content ratio of sperm suitable for fertilization is high compared to the semen before selection and there exists a deviation, in a sex ratio and hence, it is also possible to perform the solid sex selection without depending on the experience and the like.

From the results shown in Table 1 and Table 2, it is understood that, a ratio of the content of X sperm in the semen for artificial insemination prepared from the semen before selection to which the sperm preservation step is applied for a longer time is increased compared to a ratio of the content of X sperm in the semen for artificial insemination prepared from the semen before selection to which the sperm preservation step is applied for a shorter time. Data on a ratio of the content of X sperm after 6 hours elapses from a point of time that the semen is thawed in Table 2 is smaller than data on a ratio of the content of sperm X after 3 hours elapses from a point of time at which the semen is thawed. However, such a deviation is within a measurement error and hence, it is considered that the latter data is substantially equal to the former data or is increased compared to the former data.

Accordingly, it is safe to say that the above-mentioned method of preparing semen for artificial insemination is useful as a method for acquiring female individuals preferentially.

Next, a method of preparing semen for artificial insemination which can acquire male individuals or female individuals preferentially by adding a predetermined substance in the semen preservation step is explained.

In sperm, a circuit which produces energy necessary for its motion is operated using a glycolytic system in the sperm it has been known that there exists the difference in such a circuit between X sperm and Y sperm.

It is possible to prepare semen for artificial insemination which can acquire female individuals or male individuals preferentially by making use of such difference in circuit. That is, by adding a substance which impedes or accelerates any one of stages in the circuit, a motion of X sperm and a motion of Y sperm are made different from each other in advance, and semen before selection which contains such sperm is supplied to the previously-mentioned sperm screening device thus preparing semen for artificial insemination which can acquire female individuals or male individuals preferentially.

Conventionally, it has been known that there exists the difference in reaction pathway of the energy production depending on a sex chromosome which the sperm has or the like. However, it has been difficult to completely stop a motion of only one sperm out of X sperm and V sperm. That is, when a test reagent is added to sperm to an extent that the sperm completely stops a motion thereof, sperm die in many cases. Accordingly, the utilization of such a method as a method of selecting sex has not progressed.

However, with the use of the above-mentioned sperm screening device according to this embodiment, due to the performance of the device where the recovery of sperm is performed depending on a swimming speed of sperm which is one of the characteristics of the device, such a method can be used as a method of selecting sex by merely making the difference between sperm having a sex chromosome X and sperm having a sex chromosome V to an extent that only either one of the sperm becomes slightly slow, that is, with an addition of a substance with concentration extremely lower than concentration level where the substance exhibits toxicity to the sperm. Hereinafter, an experimental example is explained in detail.

### Experimental Example 3

Frozen semen of WHO OCEANIC JOVIAN ET (Holstein cattle) was thawed in accordance with the predetermined steps, 200 μl of thawed semen was sampled and was put into a tube having a capacity of 1.5 ml. Then, semen was subjected to the centrifugal separation (7 minutes, 2000 rpm), and a half of supernatant of the semen after being subjected to the centrifugal separation was thrown away and approximately 1004 of semen was left in the tube.

An approximately 100 μL of buffer solution (obtained by adding 6 mg/mL BSA+10 μg/mL of gentamycin+5 mM EGTA to the commercially available “SP-TALP” solution) is added into the tube, the buffer solution is stirred lightly and, thereafter, the buffer solution was incubated for 50 minutes at 37° with 5% CO₂.

Next, the tube was subjected to the centrifugal separation again (7 minutes, 2000 rpm), and a supernatant of the solution was thrown away, and an approximately 100 μL of buffer solution (obtained by adding 6 mg/mL BSA+10 mg/mL of gentamycin to the commercially available “SP-TALP” solution; not containing EGTA) was added into the tube and, thereafter, the solution was stirred lightly.

The tube was subjected to the centrifugal separation again (7 minutes, 2000 rpm), and 100 μL of a supernatant of the solution was thrown away, and an approximately 100 μL of buffer solution (obtained by adding 6 mg/mL BSA+10 μg/mL gentamycin +25 μM brilliant cresyl blue +5 mM glucose-6-phosphate to the commercially available “SP-TALP” solution) was added into the tube and, thereafter, the solution was incubated at 37° C. with 5% CO₂ (sperm preservation step). Then, two kinds of solutions, that is, a solution which was subjected to the incubation for 50 hours and a solution which was subjected to the incubation for 30 minutes were prepared.

Thereafter, sperm having high motility were recovered by using the sperm screening device according to this embodiment. Then, with respect to the acquired semen for artificial insemination, a ratio between X sperm and Y sperm was investigated using a real time PCR method. The result of the examination is shown in Table 3 and Table 4. Table 3 shows the result of the investigation where the incubation time was set to 50 minutes, and Table 4 shows the results of the examination where the incubation time was set to 30 minutes.

TABLE 3

| | X | Y |
|---|---|---|
| before screening | 52% | 48% |
| after screening | 35% | 65% |

TABLE 4

| | X | Y |
|---|---|---|
| before screening | 50% | 50% |
| after screening | 39% | 61% |

As can be understood from Table 3 and Table 4, by adding brilliant cresyl blue which is a phenoxazine compound to the semen before selection under a condition that an electron accepting substance is not added to the semen, it is confirmed that a ratio of sperm having sex chromosome Y contained in the semen for artificial insemination is increased compared to a ratio of sperm having a chromosome Y in the frozen semen.

To be more specific, as shown in Table 3, when the incubation time was set to 50 minutes, while a ratio of X sperm and a ratio of Y sperm were 52% and 48% respectively in the frozen semen, a ratio of X sperm and a ratio of Y sperm in the acquired semen for artificial insemination were 35% and 65% respectively.

Further, as shown in Table 4, when the incubation time was set to 30 minutes, while a ratio of X sperm and a ratio of Y sperm in the frozen semen were 50% and 50% respectively, a ratio of X sperm and a ratio of Y sperm in the acquired semen for artificial insemination were 39% and 61% respectively.

As has been described heretofore, it is found that the method of preparing semen for fertilization according to this embodiment can change a ratio of X sperm or a ratio of Y sperm in the semen for artificial insemination by adding the above-mentioned predetermined substance to the semen.

As has been described above, the sperm selection unit structure according to this embodiment includes: the storage part which stores semen before selection and containing sperm acquired from an animal therein; the buffer solution flow passage which communicates with the storage part and in which a buffer solution flows in the direction toward the storage part as a laminar flow; and the swum-up passage which communicates with the storage part such that the sperm swim up in the buffer solution flow passage from the storage part, wherein the downstream part of the swim-up passage is formed of the flow passage having a wide width where the buffer solution flows as a slow flow while maintaining a laminar flow state, and the downstream end portion of the flow passage is opened in a state where the downstream end portion faces the wall surface of the storage part. With such a configuration, it is possible to provide the sperm selection unit structure capable of efficiently separating sperm having favorable motility.

Further, the sperm screening device according to this embodiment is characterized in that, in the sperm screening device which allows sperm recovered from an animal to swim up in a buffer solution in a laminar flow state in the predetermined zone, and includes the sperm selection unit structure according to this embodiment which recovers sperm which succeed in swimming up, the recovery passage for recovering sperm which succeed in swimming up and the buffer solution supply passage for supplying a buffer solution which flows downwardly in the swim-up passage and the recovery passage are connected to an upstream end of the swim-up passage, while a portion of the buffer solution supplied from the buffer solution supply passage is made to flow downwardly into the swim-up passage, a remaining portion of the buffer solution is made to flow downwardly into the recovery passage so as to form a recovering flow thus recovering the sperm which succeed in swimming up in the recovering flow. With such a configuration, it is possible to recover sperm having favorable motility extremely efficiently.

Further, the method of preparing semen for fertilization according to this embodiment is the method of preparing semen for fertilization using the abovementioned sperm screening device, and the method includes: the sperm adding step of adding the semen before selection to the storage part; the sperm swim-up step of making sperm contained in the semen before selection added to the storage part swim up through the swim-up passage by making a buffer solution flow through the buffer solution flow passage and the recovery passage from the buffer solution supply passage at a flow speed which exceeds motility of the sperm which succeed in swimming up with respect to the recovery passage and at a flow speed which exceeds a limit flow speed by which sperm having motility but being not desired to be recovered are swimmable up and is lower than a limit flow speed by which sperm having favorable motility are swimmable up and at which a laminar flow is formed in the swim-up passage with respect to the buffer solution flow passage; and the recovery step of recovering the sperm which succeed in swimming up and are captured by the recovering flow thus preparing for fertilization. With such a configuration, it is possible to provide a method of preparing semen for fertilization which contains a large amount of sperm having favorable motility.

Finally, the respective embodiments described above are merely one example of the present invention, and the present invention is not limited to the abovementioned embodiments. Accordingly, it is needless to say that, besides the configurations of the respective embodiments described above, various modifications are conceivable depending on designs provided that the modifications fall within the technical concept of the present invention.

### EXPLANATION OF SYMBOLS

10: storage part
11: narrow swim-up passage
12: laminar flow passage
13: slit-shaped opening portion
20: storage part
21: narrow swim-up passage
22: laminar flow passage
23: slit-shaped opening portion
30: storage part
31: narrow swim-up passage
32: laminar flow passage
33: slit-shaped opening portion
42: chip body
46: storage part 46 for storing semen before selection
52: micro flow passage
61: buffer solution supply passage
62: merging portion
63: swim-up passage
64: recovery passage
65: narrow swim-up passage
66: laminar flow passage
67: slit-shaped opening portion
A: selection unit structure
B: selection unit structure
C: selection unit structure
D: sperm screening device
S: sperm

1. A sperm selection unit structure comprising;
a storage part which stores semen before selection and containing sperm acquired from an animal therein;
a buffer solution flow passage which communicates with the storage part and in which a buffer solution flows in the direction toward the storage part as a laminar flow; and
a swim-up passage which communicates with the storage part such that the sperm swim up in the buffer solution flow passage from the storage part, wherein
a downstream part of the swim-up passage is formed of a flow passage having a wide width where the buffer solution flows as a slow flow while maintaining a laminar flow state of the buffer solution, and a downstream end portion of the flow passage has an opening facing a wall surface of the storage part.

2. The sperm selection unit structure according to claim 1, wherein the flow passage having a wide width n arrows toward upstream from the opening.

3. The sperm selection unit structure according to claim 1, wherein a plurality of swim-up passages are connected to the storage part.

4. A sperm screening device provided with the sperm selection unit structure according to claim 1 where sperm acquired from an animal are made to swim up through the buffer solution in the buffer solution flow passage in which the buffer solution is flowing in a laminar flow state, further comprising:
a recovery passage which recovers the sperm which succeed in swimming up and a buffer solution supply passage which supplies the buffer solution which is made to flow down in the swim-up passage, wherein the recovery passage is connected to an upstream end of the swim-up passage,
and wherein the device is configured to that a portion of the buffer solution supplied from the buffer solution supply passage flows down into the swim-up passage, and a remaining portion of the buffer solution flows down into the recovery passage thus forming a recovering flow, whereby the sperm which succeed in swimming up are recovered in the flow of the recovering flow.

5. The sperm screening device according to claim 4, wherein the buffer solution supply passage has allow passage cross-sectional area configured to provide a flow rate of the buffer solution which enables the recovering flow to have a flow speed exceeding a swim-up ability of the sperm which succeed in swimming up through the recovering flow.

6. A method of preparing semen for fertilization using the sperm screening device according to claim 5, the method comprising:
a sperm adding step of adding the semen before selection to the storage part;
a sperm swim-up step of making sperm contained in the semen before selection added to the storage part swim up through the swim-up passage by making the buffer solution flow from the buffer solution supply passage buffer solution flow passage and the recovery passage at a flow speed which exceeds motility of the sperm which succeed in swimming up with respect to the recovery passage and at a flow speed which exceeds a limit flow speed by which sperm having motility but being not desired to be recovered are swimmable up and is lower than a limit flow speed by which sperm having favorable motility are swimmable up and at which a laminar flow is formed in the swim-up passage with respect to the buffer solution flow passage, and
a recovery step of recovering the sperm which succeed in swimming up and are captured by the recovering flow thus preparing semen for fertilization.

7. The method of preparing semen for fertilization according to claim 6, wherein using timing at which sperm having a sex chromosome X and contained in the semen before selection acquire fertility as a threshold time, a ratio of sperm having a sex chromosome Y contained in the semen for fertilization is increased by performing the sperm adding step before the threshold time elapses, or a ratio of sperm having a sex chromosome X and contained in the semen for fertilization is increased by performing the sperm adding step after the threshold time elapses.

8. The method of preparing semen for fertilization according to claim 6, wherein in a sperm preservation step in which the semen before selection is preserved before the sperm adding step is performed, among sperm in the semen before selection, motility of sperm having either one of sex chromosomes out of the sperm having the sex chromosome X and the sperm having the sex chromosome V is enhanced so that the swim-up of the sperm having either one of the sex chromosomes in the swim-up passage is accelerated in the sperm swim-up step whereby a ratio of sperm having either one of sex Chromosomes and contained in the semen for fertilization acquired by the recovery step is increased.

9. The method of preparing semen for fertilization according to claim 6, wherein in a sperm preservation step in which the semen before selection is preserved before the sperm adding step is performed, among sperm in the semen before selection, motility of sperm having either one of sex chromosomes out of sperm having sex chromosome X and sperm having sex chromosome Y is lowered so that the swim-up of sperm having either one of sex chromosomes in the swim-up passage is suppressed in the sperm swim-up step whereby a ratio of sperm having the other sex chromosome and contained in the semen for fertilization acquired in the recovery step is increased.

10. The method of preparing semen for fertilization according to claim 6, wherein a preservation time in a sperm preservation step in which the semen before selection is preserved before the sperm adding step is performed is prolonged so that a ratio of sperm having sex chromosome X and contained in the semen for fertilization is increased.

11. The method of preparing semen for fertilization according to claim 6, wherein in a sperm preservation step in which the semen before selection is preserved before the sperm adding step is performed, a phenoxazine compound and an electron accepting substance are present in the semen before selection so that a ratio of sperm having sex chromosome X and contained in the semen for fertilization is increased.

12. The method of preparing semen for fertilization according to claim 6, wherein in a sperm preservation step in which the semen before selection is preserved before the sperm adding step is performed, a phenoxazine compound is present in the semen before selection so that a ratio of sperm having sex chromosome Y contained in the semen for fertilization is increased.

13. The method of preparing semen for fertilization according to claim 12, wherein the electron accepting substance is at least one selected from a group consisting of flavins, phenazines, and NAD(P)H oxidoreductase.

14. The method of preparing semen for fertilization according to claim 11, wherein in the sperm preservation step, hexose-6-phosphate is further added to the semen before selection.

15. The method of preparing semen for fertilization according to claim 14, wherein, the hexose-6-phosphate is one or a mixture of two or more selected from a group consisting of D-glucose 6-phosphate, fructose-6-phosphate, mannose-6-phosphate and galactose-6-phosphate.

16. The method of preparing semen for fertilization according to claim 11, wherein the phenoxazine compound is one or a mixture of two or more selected from a group consisting of brilliant cresyl blue, nile blue, basic blue and meldola blue.
